(19) United States
(12) Patent Application Publication
(10) Pub. No.: US 2006/0100572 A1
(43) Pub. Date: May 11, 2006

(54) Dialysis catheter tip and method of manufacture

(76) Inventors: Kristian DiMatteo, Waltham, MA (US); Todd Beaupre, Reading, MA (US); James Culhane, Westborough, MA (US); Benjamin Bell, Haverhill, MA (US); James Weldon, Roslindale, MA (US)

Correspondence Address: FAY KAPLUN & MARCIN, LLP, 15O BROADWAY, SUITE 702, NEW YORK, NY, 10038, US

(21) Appl. No.: 11/266,925

(22) Filed: Nov. 4, 2005

Related U.S. Application Data
(63) Continuation-in-part of application No. 10/777,545, filed on Feb. 12, 2004.

(51) Int. Cl. A61M 3/00

(52) U.S. Cl. 604/43; 264/109; 604/264

(57) ABSTRACT

A multi-lumen catheter comprising a first lumen extending through the catheter to a first distal opening, and a second lumen extending through the catheter to a second distal opening distal to the first distal opening so that an extending portion of a septum separating the lumens extends distally past the first distal opening. A tip is overmolded on the extending portion and includes a first ramp adjacent to the first distal opening and a second ramp adjacent to the second distal opening. The ramps direct fluids exiting the openings away from a longitudinal axis of the catheter.

### PRIORITY CLAIM/INCORPORATION BY REFERENCE

The present application claims priority to U.S. patent application Ser. No. 10/777,545 entitled “Dialysis Catheter Tip” naming as inventor Kristian DiMatteo which was filed Feb. 12, 2004. The entire disclosure of this application is expressly incorporated herein by reference.

### BACKGROUND OF THE INVENTION

Medical procedures for the treatment of chronic diseases often require repeated access to the vascular system for the injection of therapeutic compounds and the sampling of blood. Kidney dialysis, chemotherapy and other chronic treatments generally rely on catheters for both injection to and withdrawal of fluids from the vascular system. For example, during kidney dialysis, large amounts of blood are withdrawn from the patient, treated externally in a dialysis machine to remove impurities and add nutrients, medications and other therapeutic elements and returned to the patient.

Typically, a single catheter having two or more lumens is used for the removal and return of the blood with a first of the lumens being used to aspire impure blood from a blood vessel (usually a vein) and a second of the lumens being used to return the treated blood to the blood vessel. A single catheter tip including inlet and outlet orifices connected to the first and second lumens, respectively, is commonly used to perform both functions.

Since the inlet and outlet orifices are located on the same tip, a portion of the treated blood exiting the outlet orifice is recirculated directly through the inlet orifice to the dialysis machine. This delays treatment of portions of the venous blood displaced by the recirculated fluid, increasing the time required to achieve a desired amount of purification, as well as the cost of the procedure and patient discomfort.

### SUMMARY OF THE INVENTION

In one aspect, the present invention is directed to a multi-lumen catheter comprising a first lumen extending through the catheter to a first distal opening, and a second lumen extending through the catheter to a second distal opening which is distal to the first distal opening so that an extending portion of a septum separating the lumens extends distally past the first distal opening. A tip is overmolded on the extending portion and includes a first ramp adjacent to the first distal opening and a second ramp adjacent to the second distal opening. The ramps direct fluids exiting the openings away from a longitudinal axis of the catheter.

The present invention is further directed to a method of forming a distal tip for a multi-lumen catheter whereby a catheter is provided with a first lumen extending through the catheter to a first distal opening and a second lumen extending through the catheter to a second distal opening which is distal to the first distal opening so that an extending portion of a septum separating the lumens extends past the first distal opening. A tip bonded to the extending portion includes a first ramp adjacent to the first distal opening and a second ramp adjacent to the second distal opening. The first and second ramps direct fluids exiting from the first and second distal openings at first and second angles relative to a longitudinal axis of the catheter.

### BRIEF DESCRIPTION OF THE DRAWINGS

FIG. 1 is a side elevation view of a dual lumen catheter according to an embodiment of the present invention;

FIG. 2 is a perspective view of the dual lumen catheter shown in FIG. 1;

FIG. 3 is a cross sectional view showing the elongated body of the catheter along line III-III;

FIG. 4 is a cross sectional view showing the catheter along line IV-IV;

FIG. 5 is a schematic diagram showing the fluid flow through the catheter according to an embodiment of the invention in a normal mode;

FIG. 6 is a schematic diagram showing the fluid flow exiting the catheter of FIG. 5 in a reverse mode;

FIG. 7 is a cross sectional side elevation view of an intermediary step in the construction of a catheter tip according to a different embodiment of the invention;

FIG. 8 shows a top plan view of the distal portion of the intermediary step shown in FIG. 9;

FIG. 9 shows a front elevation view of the distal portion of the intermediary step shown in FIG. 8;

FIG. 10 shows a cross sectional side elevation view of a different embodiment of the catheter tip according to the invention;

FIG. 11 shows a side elevation view of an alternative exemplary manufacturing method for a catheter tip according to the invention;

FIG. 12 shows a side elevation view of another alternative manufacturing method for a catheter tip according to the invention;

FIG. 13 is a side elevational view of an exemplary embodiment of a compound curve slope of a ramp in front of an arterial lumen opening of a catheter according to the present invention;

FIG. 14 is a side elevational view of the catheter of FIG. 14 illustrating fluid flow patterns;

FIG. 15 is a side elevational view of a portion of a hemodialysis catheter embodying features of another exemplary embodiment of the invention;

FIG. 16 is a bottom plan view of the catheter of FIG. 15;

FIG. 17 is a top plan view of the catheter of FIG. 14;

FIG. 18 is a longitudinal sectional view taken along line 13-13 of FIG. 17;

FIG. 19 is a cross-sectional view taken along line 16-16 of FIG. 16;

FIG. 20 is a cross-sectional view taken along line 17-17 of FIG. 17;

FIG. 21 is a cross-sectional view taken along line 18-18 of FIG. 17;

FIG. 22 is another top plan view of the catheter of FIG. 15 illustrating fluid flow patterns which are produced;

FIG. 23 is a cross-sectional view taken along line 20-20 of FIG. 22;

FIG. 24 is a side elevational view of the catheter and fluid flow patterns seen in FIG. 22;

FIG. 25 is a top plan view of a distal end of the catheter of FIG. 15;

FIG. 26 is a bottom plan view to the catheter seen in FIG. 25;

FIG. 27 is a longitudinal sectional view taken along line 24-24 of FIG. 25;

FIG. 28 is a cross-sectional view taken along line 25-25 of FIG. 25;

FIG. 29 is another top plan view of the catheter of FIG. 15;

FIG. 30 is another side elevational view of the catheter of FIG. 15;

FIG. 31 is a sectional view taken along line 28-28 of FIGS. 29 and 30;

FIG. 32 is a sectional view taken along line 29-29 of FIGS. 29 and 30;

FIG. 33 is a sectional view taken along line 30-30 of FIGS. 29 and 30;

FIG. 34 is a sectional view taken along line 31-31 of FIGS. 29 and 30;

FIG. 35 is a sectional view taken along line 32-32 of FIGS. 29 and 30;

FIG. 36 is a longitudinal sectional view through the catheter of FIG. 15 as the bolus is insert molded onto the distal end of the tube;

FIG. 37 is a side elevational view of a portion of a tunneling tool that is used to pull a catheter tip and a catheter tube through a subcutaneous tunnel;

FIG. 38 is a top view of the tunneling tool;

FIG. 39 is a longitudinal cross-sectional view of the tunneling tool after it is inserted into a venous lumen of the catheter tube; and

FIG. 40 is a side elevational view of the catheter tube and the tunneling tool secured together with an oversleeve and ready to be pulled through a tunnel.

### DETAILED DESCRIPTION

The present invention may be further understood with reference to the following description and the appended drawings, wherein like elements are referred to with the same reference numerals. The present invention relates to devices for accessing the vascular system. Although the present invention is described in regard to a catheter used to withdraw and return blood during dialysis, those skilled in the art will understand that the invention is equally applicable to any treatment in which a single catheter to withdraw fluid from and provide fluid to a blood vessel or other lumen. More particularly, the invention relates to catheter tips that minimize recirculation during such treatments.

To reduce recirculation, the tips of conventional dialysis catheters are shaped, to a certain extent, to separate the inlet and outlet orifices. For example, conventional designs have staggered orifices, with the outlet orifice further downstream (in the direction of the flow of blood) than the inlet orifice. Typically, in this configuration, the outlet orifice is placed on the tip distally of the inlet orifice. However, at times it is necessary to reverse the direction of flow through the catheter so that the inlet orifice serves as an outlet and the outlet orifice serves as an inlet.

In this reverse mode, the outlet orifice is no longer downstream of the inlet, increasing recirculation. This effect is alleviated to a certain extent by the flow of blood which tends to entrain the injected blood away from the catheter tip. However, the flow of blood pulsates with the beating heart and, when the rate of flow is at its lowest, the purified blood exiting the conventional catheter is not entrained away from the tip and the inlet through which it may be recirculated.

To gain a quantitative understanding of the scope of the problem caused by recirculating blood, exemplary recirculation rates determined experimentally are described below. For an exemplary conventional staggered tip catheter with inlet and outlet orifices displaced longitudinally relative to one another, the recirculation rate in the normal more of operation is about 0.4% while for the reverse mode of operation the recirculation rate is about 20.9%. In contrast, exemplary embodiments of a catheter tip according to the present invention provide recirculation rates in the normal mode of between about 0.4% and 2.4%, with reverse mode recirculation rates of between about 6.3% and about 7.8%. As can be seen, the exemplary embodiments according to the present invention provide a substantial reduction in recirculation in the reverse mode of operation of the catheter, while maintaining normal mode recirculation comparable to that of the conventional catheters.

In addition to the amount of recirculation in both reverse and normal modes of operation, thrombogenicity of the design is of interest. This refers to the tendency of the catheter tip to facilitate coagulation of the blood flowing therethrough forming coagulated particles known as thrombi. As is understood by those skilled in the art, thrombi may be very dangerous if they become dislodged and travel through the body. The hemolysis of the catheter tip (i.e., the tendency of the tip to damage blood cells flowing therethrough) is also important.

The exemplary embodiments of the present invention thus provide improvements in the ability of the catheter to minimize recirculation in a reverse mode of operation, while at the same time retaining the ability to minimize recirculation in the normal mode of operation. Those skilled in the art will understand that this latter property is important as the catheter spends a majority of its operational life in the normal mode of operation with the reverse mode of operation being implemented less frequently. In addition, the embodiments of the catheter tip according to the present invention retain acceptable thrombogenicity and hemolysis properties.

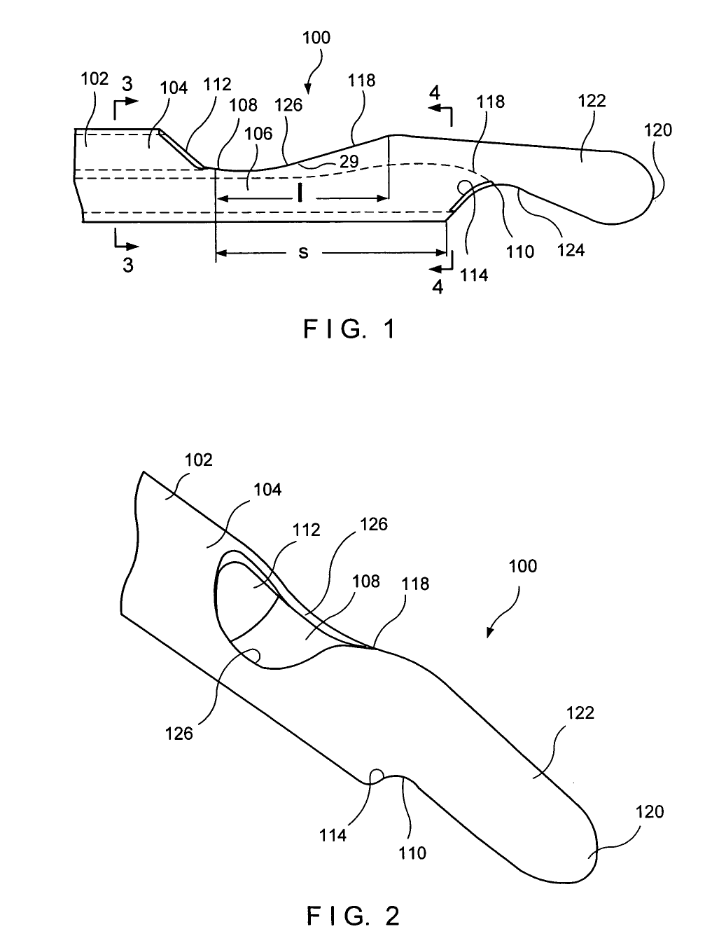

FIGS. 1 and 2 depict a tip 100 for a dialysis catheter (not shown) comprising a proximal substantially tubular portion 102 providing a transition to the elongated tubular body of the catheter as will be described below. The tip 100 reduces recirculation in the reverse mode through a novel shaping of first and second openings 108, 110 which, in the normal mode of operation, act respectively as inlet and outlet openings of the catheter. Additional control over recirculation is gained by providing in the tip 100 a flow control element 122 shaped to achieve one or more of several goals. For example, the flow control element 122 may be designed to deflect flow from the first opening 108 away from the tip 100, and particularly away from the second opening 110. In the reverse mode of operation this feature minimizes an amount of flow exiting the first opening 108 ingested by the second opening 110. The flow control element 122 may also be designed to reduce recirculation in the normal mode by deflecting fluid exiting the second opening 110 away from the first opening 108.

In addition to FIGS. 1-4, the normal and reverse modes of operation of the tip 100 are depicted in FIGS. 5 and 6. FIG. 5 shows the normal mode where a second lumen 106 of the tip 100 which is connected fluidly with the second (outlet) opening 110, ejects fluid into the bloodstream traveling in the direction shown by the arrow B. Aspiration of untreated blood occurs through the first (inlet) opening 108 which is connected to a first lumen 104 of the tip 100. FIG. 6 shows the reverse mode, where the first lumen 104 and the first opening 108 inject fluid into a vein, while the second lumen 106 and the second opening 110 are used to aspire blood therefrom. As will be described in greater detail below, the location and shape of the first and second openings 108, 110 and a ramp 118 described in more detail below, as well as the shape of the flow control element 122, cooperate to obtain desired characteristics of the catheter tip 100.

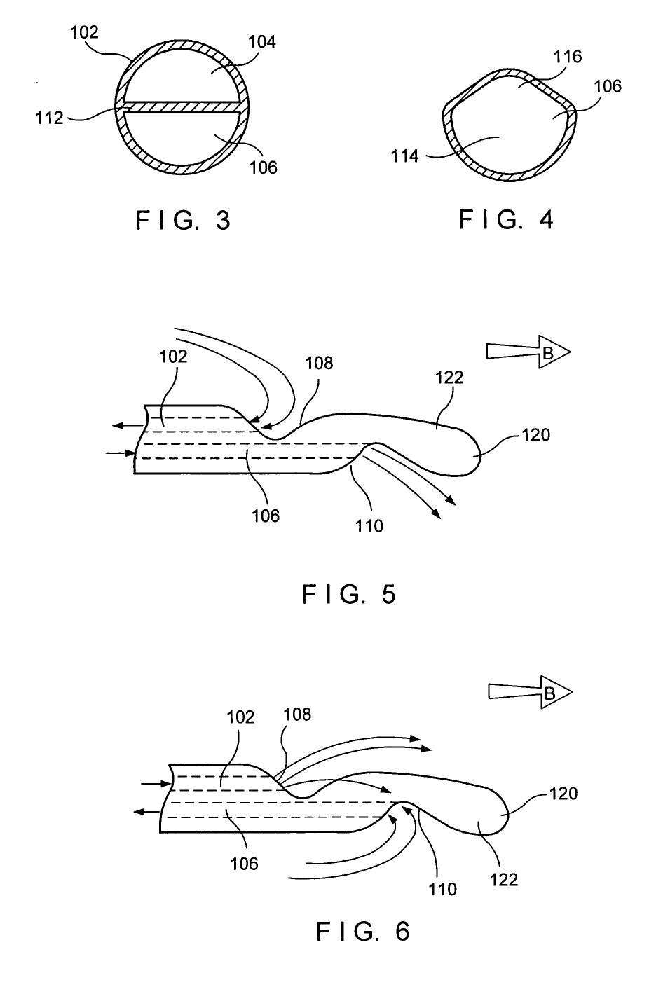

FIG. 3 shows a cross sectional area along line III-III of the proximal portion 102 of the catheter tip 100 near a location where the tip 100 transitions to the elongated body of the catheter. The first and second lumens 104 and 106 are shown in an exemplary configuration, each having a substantially ‘D’ shaped cross section. This configuration is compatible with a conventional catheter having a circular cross section and two lumens of approximately equal dimensions. It will be apparent to those skilled in the art that different cross sectional shapes may be used in the proximal portion 102 of the tip 100 depending on the shape of the catheter and the shapes of the lumens therein. Different methods of connecting or integrating the tip 100 into the catheter may also be used, as will be described below.

In greater detail, the flow control element 122 includes a ramp 118 near the first opening 108, as shown in FIG. 1. The ramp 118 is preferably oriented so that in the reverse mode, fluid exiting the first opening 108 is deflected upward, away from the main body of the tip 100. Those skilled in the art will understand that, in this context, the directions “up” and “down” are used simply in relation to the orientation of the drawings and do not refer to the orientation of any features when in use. The actual orientation of the components of the tip 100 may be similar, inverted, or shifted sideways relative to the orientation shown. The ramp 118 preferably has a length l selected to provide a desired deflection of the flow. Similarly, the ramp 118 preferably has a ramp angle α also selected to obtain a desired deflection. The angle α may be constant throughout the length of the ramp 118 or may be vary therealong. As would be understood by those skilled in the art, the specific shape, length l and angle α of the ramp 118 may be selected based on the application for which a catheter including the tip 100 is intended. For example, these characteristics may be varied based on expected blood flow rate, inlet and outlet flow rate, desired performance of the catheter in the normal and reverse modes of operation as well as based on the characteristics of the intended anatomical location of the catheter. For example, different cavities and/or lumens will have different fluid flow patterns and the design may be varied accordingly. More specifically, the ramp 118 may have a shape that is substantially planar or which is curved, for example in either a convex or concave shape. For example, the angle α may preferably be between 150° and 175° and is more preferably approximately 1650.

The flow control element 122 may also include lateral elements 126 designed to prevent flow from “wrapping” around the sides of the tip 100 toward the second opening 110. The first opening 108 includes an orifice 112 formed on a plane diagonal to a longitudinal axis of the first lumen 104. The specific angle and size of the orifice 112 is preferably selected to cooperate with the ramp 118 to obtain a selected flow rate out of the first opening 108. The length of the flow control element 122 in front of the ramp 118 may also be selected in part to reduce the tendency of blood to recirculate during the reverse mode. In addition, a contoured bolus 120 may be provided at a distal-most point of the tip 100 to facilitate insertion of the tip/catheter assembly into the vein and to assist in navigating the assembly therein. Preferably, the contoured bolus 120 forms an atraumatic tip for catheter tip 100 allowing the catheter tip 100 to penetrate and navigate within the blood vessels without causing injury thereto.

Another important consideration in the design of the catheter tip 100 is the stagger distance s between the first and second openings 108, 110. An increase in the stagger distance s generally reduces recirculation. However, an excessive increase in the stagger distance s may make the catheter tip 100 impractical for use in a blood vessel (i.e., the length of the tip 100 may make navigation difficult or impossible). Accordingly, an optimum stagger distance s may be determined for various applications. For example, the stagger distance s for a dialysis catheter of typical dimensions is preferably between about 1.5 cm to about 2.5 cm, while for applications in vessels of greater or lesser diameter and with longer or shorter radii of curvature, different optimum dimensions may be arrived at.

Additional control of the flow surrounding the tip 100 may be achieved by forming the flow control element 120 with a second ramp 124 designed to deflect flow exiting the second opening 110 in the normal mode. The second ramp 124 or a similar flow control device may be used to further reduce recirculation in the normal mode by directing the exiting flow away from the first opening 108. For example, the second ramp 124 preferably has a length and a ramp angle β designed to cooperate with the orifice 114 of the second opening 110. For example the orifice 114 may be formed on a plane inclined with respect to a longitudinal axis of the second lumen 106 to form a substantial mirror image of the orifice 112 of the first opening 108. Properly forming the contours of the second ramp 124 further reduces recirculation in the normal mode. However, the design of the second opening 110 and the second ramp 124 is generally less critical than that of the first opening 108 and the first ramp 118 as, in the normal mode of operation, flow exiting the second opening 110 is entrained away from the first opening 108 by the natural flow of blood and is less likely to be recirculated.

The flow control element 122 may also include features adapted to increase an exit plane cross sectional area of the second opening 110. For example, an upper expanded section 116 may be included in the design, as shown in FIGS. 1 and 4. The upper expanded section 116 forms a bulge or expansion of the second lumen 106, in a region near the orifice 114. The purpose of the upper expanded section 116 is to increase the cross sectional area at the exit of the second lumen 106 to reduce the velocity of the blood flow exiting the second opening 110 in the normal mode of operation. A lower outflow velocity reduces the possibility of damage to adjacent tissue. Accordingly, providing an upper expanded section 116 or a similar structure allows for a high flow rate exiting the dialysis catheter while reducing the flow velocity.

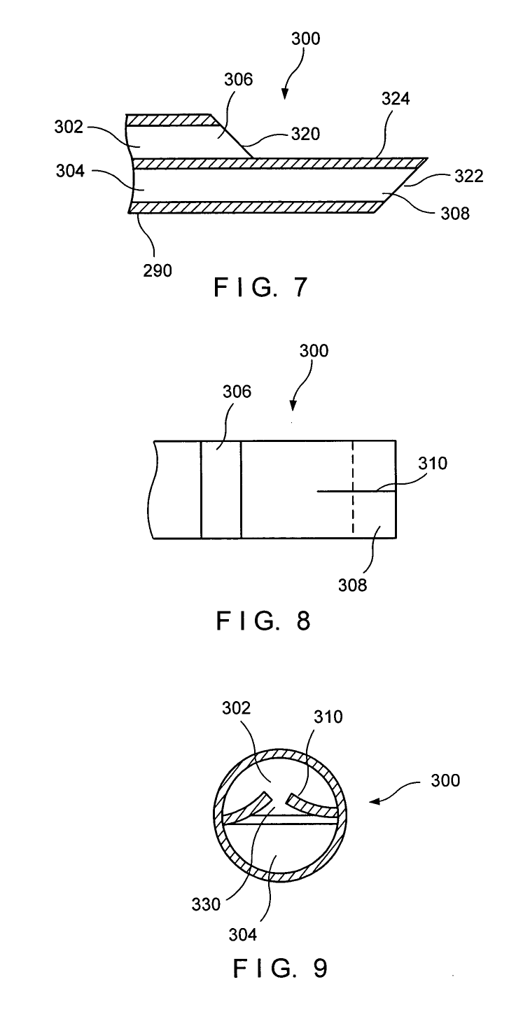

The tip structure may be formed in multiple steps. For example, in one embodiment the catheter shaft extends into a catheter tip 300, and is shaped to form a core of the tip 300. An overmolding process may then be used to form the contoured bolus defining the flow control elements of the tip, according to the invention. As shown in FIGS. 7-10, the catheter tip 300 is formed by modifying the distal end of a catheter 290 and attaching thereto a small, separately formed element. In this exemplary assembly method, it is not necessary to mold the entire tip 300 as a separate unit for later attachment to the catheter 290.

FIG. 7 shows the tip 300 of the catheter 290 in an initial step of fabrication. The distal portion of the catheter 290 is trimmed, for example, skived, to obtain a staggered configuration of the openings. In the exemplary embodiment, the first lumen 302 is cut along a plane 320, at a selected angle with a portion of the first lumen 302 distal of the plane 320 removed such that a top surface 324 of the second lumen 304 is exposed. The second lumen 304 is cut along a plane 322 which may be, for example, at an angular orientation opposite to that of the plane 320. In this manner the first orifice 306 and the second orifice 308 are formed so that they point towards opposite sides of the tip 300. Alternatively, other manufacturing methods suitable to obtain the first and second orifices 306, 308 in the staggered configuration shown may be used. Thus, the catheter 290 may be shaped during manufacture to have a distal end with staggered lumens.

A slit or web cut 310 may be formed in a subsequent step, along the distal end of an upper surface 324 for a length selected to allow upward expansion of the second lumen 308, to form an upper expanded section 330 in a subsequent forming step. As discussed above, the upper expanded section 330 lowers the velocity of the flow exiting the second orifice 308 in the normal mode, by providing a larger exit plane cross sectional area of the second lumen 304. By cutting the slit 310 in the upper surface 324, a molding core or other tool may be inserted in the distal portion of the second lumen 304 to expand the distal portion upward. The size of the slit 310 is preferably based, for example, on the material of which the catheter 290 is formed, on a desired maximum exit velocity of the flow leaving the second lumen 304 and a desired volume flow rate.

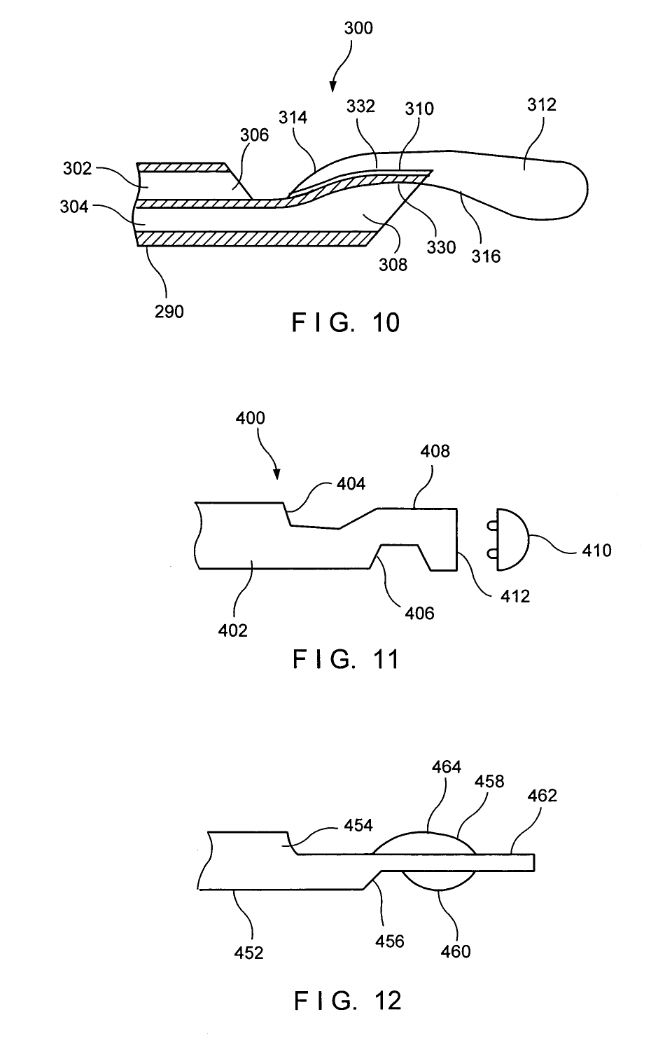

FIG. 10 shows a later step in the formation of the distal tip 300 of the catheter 290. Here, a contoured bolus 312 is formed by overmolding an upper surface 324 of the second lumen 304. In the exemplary embodiment, the molding process attaches the contoured bolus 312 to the catheter 290, and also forms the upper expanded section 330 by opening up the slit 310. According to this exemplary embodiment, the contoured bolus 312 defines a first ramp 314 designed to control and direct the flow exiting the first orifice 306, in the reverse mode. The contoured bolus 312 may also define a second ramp 316 adapted to deflect and control the flow exiting the orifice 308, in the normal mode. All the features described above with reference to different embodiments of the distal tip may be included in the flow deflection element 332 defined by the contoured bolus 312. Accordingly, the present embodiment also achieves a significant reduction in fluid recirculation in both the normal and the reverse modes of operation.

As shown in FIG. 11, a distal tip 400 according to a different embodiment of the invention is assembled from multiple parts. A catheter 402 is provided with a first orifice 404 and a second orifice 406 by skiving or by any other known manufacturing process. The same process may also form a flow deflection element 408 at the distal end of catheter 402. A tip 410 may be formed separately, by molding, grinding or any other suitable process and then attached to a distal surface 412 of the catheter 402. The exemplary method results in a distal tip 400 comprising flow deflection portions for both the orifices 404 and 406, as well as a tip portion 410 shaped to facilitate insertion and navigation in the blood vessels.

FIG. 12 shows yet another exemplary embodiment of a manufacturing process used to form an improved distal tip 450 of a catheter, such as a dialysis catheter. In this example, the catheter 452 is skived to obtain a staggered configuration of the first and second orifices 454 and 456 and an extension 462 of a portion of the catheter 452 is left after skiving to provide a base upon which a flow control portion of the tip 450 is formed. It will be apparent to those of skill in the art that other manufacturing methods in addition to skiving may be employed to obtain a distal end of the catheter 452 as shown in FIG. 12 The extension portion 462 may be melted, for example, by applying RF energy thereto, in conjunction with other shaping and/or grinding to obtain the final shape of the flow control element 464 including, for example, flow control ramps for both the first orifice 454 and the second orifice 456, as well as any or all of the other features described above with respect to other embodiments.

Various other considerations may affect the details of the design and construction of the improved catheter tip according to embodiments of the invention. For example, the tip should not cause a sudden jump in the outer diameter of the catheter, which make the device unsuitable for certain applications. Accordingly, a maximum radial dimension of the tip is preferably substantially the same or smaller than the radius of the distal portion of the catheter to which the tip is attached. Similarly, the tip portion is designed so that it does not restrict the passage of the catheter through an introducer sheath. The tip also is designed to prevent obstructing the passage of a guidewire through the catheter. A guidewire that may be used with the base catheter is thus also usable with the catheter plus the distal tip. Embodiments of the distal tip also do not increase the pressure required to pass fluid therethrough. Thus, no changes are required to the supporting equipment. In addition, the improved tip has hemolysis and thrombogenesis characteristics comparable with those of conventional catheters.

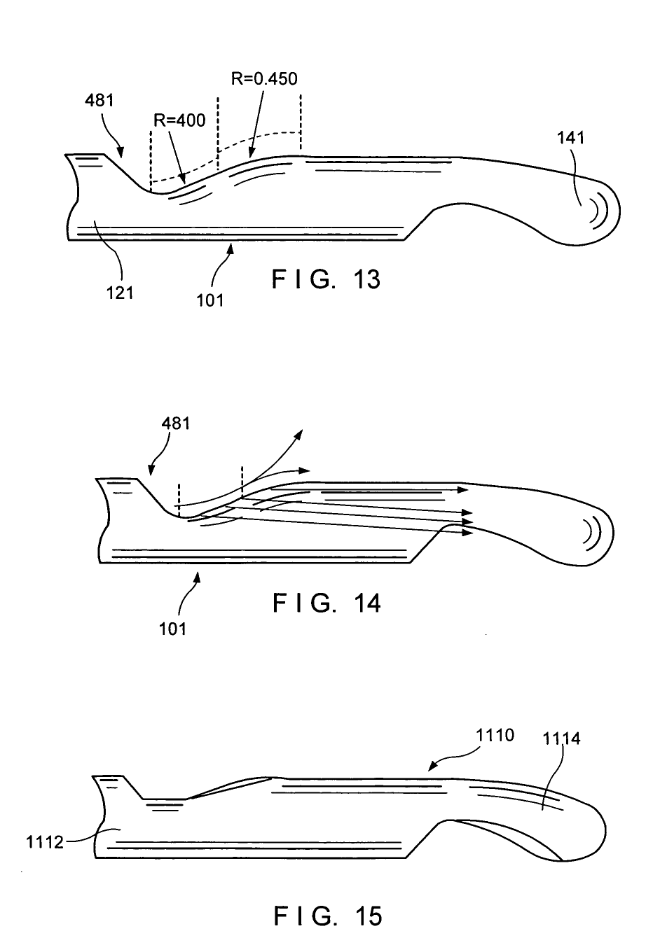

FIGS. 13 and 14 show the catheter 101 and the ramp 601 which, with the open and skived end 321 of the arterial lumen 221, forms an arterial port 481. FIG. 13 illustrates ramp angles and FIG. 14 illustrates fluid flow patterns generated as a function of the ramp angles.

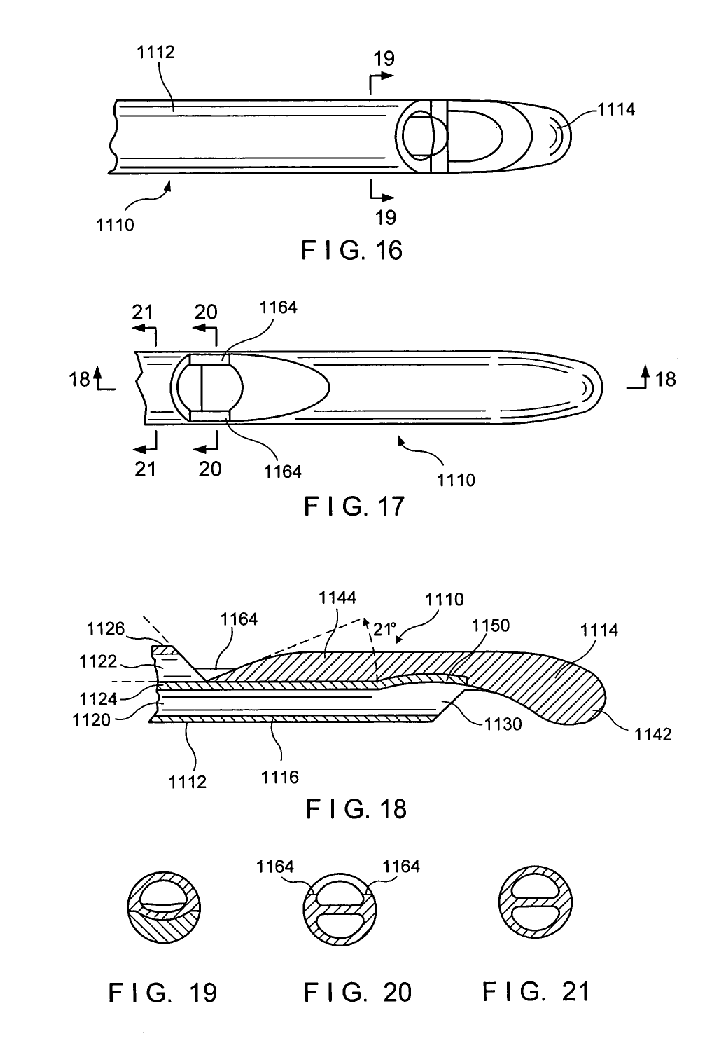

Turning now to FIGS. 15-25, a further exemplary embodiment of a dual lumen catheter according to the present invention is shown generally at 1110. The catheter 1110 comprises a catheter tube 1112 onto which a bolus tip 1114 is insert molded.

The catheter tube 1112 comprises a tube body 1116 (see FIG. 18) which contains a venous lumen 1120 and an arterial lumen 1122 separated by a septum 1124. The lumens 1120, 1122 and the septum 1124 are enclosed by a body wall 126 which in this embodiment is substantially cylindrical.

As best seen in FIG. 18, the venous lumen 1120 has a distal end 1130 cut off (skived) at a predetermined angle (e.g., about 45°) relative to the septum 1124. The arterial lumen 1122 has a distal end 1132 displaced a predetermined longitudinal distance from the end 1130 of the venous lumen 1120 and also cut off (skived) at a predetermined angle (e.g., about 45°) relative to the septum 1124. A surface 1134 of the septum 1124 then forms an outer surface of the tube 1112 between the ends 1130 and 1132. The tube 1112 includes side walls 1136 which bracket the surface 1134, as shown in FIGS. 27 and 28. In a preferred embodiment, the side walls 1136 are created by skiving the outer tube 112 and extend upward from the septum by a height “W” as shown in FIG. 28. The height W of the side walls 1136 according to this embodiment is preferably between approximately 0.015 and 0.035 inches.

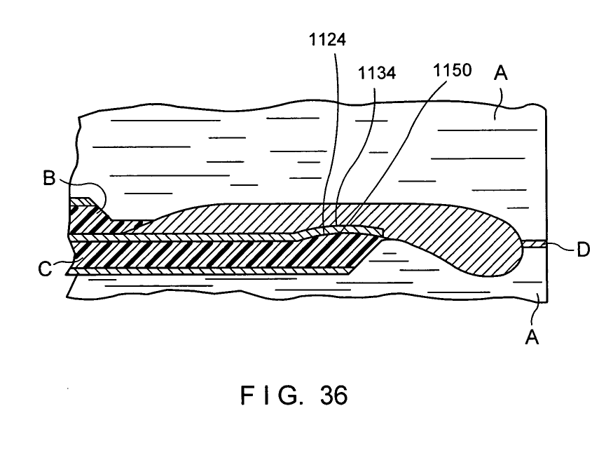

Referring to FIG. 36, the bolus tip 1114 is insert molded onto the tube 1112 in a conventional manner with mold halves forming each side of the catheter. Before the mold halves are closed over the tube 1112, an insert pin B is placed in the arterial lumen 1122, and an insert pin C is inserted into the end 1130 of the venous lumen 1120. The pin C has a bulbous center section which stretches the septum 1124 upwardly and outwardly adjacent its free end, at 1150. Molten plastic is then introduced into the closed mold halves through a gate D which may be formed anywhere in either mold half. In a preferred embodiment, the gate D is formed in a top mold half or a top of the mold.

The molten plastic adheres to the surface 1134 of the septum 1124 and to the side walls 1136. The bulge 1150 formed in the thermoplastic septum 1124 retains this shape when the dies A and B and the pin C are removed.

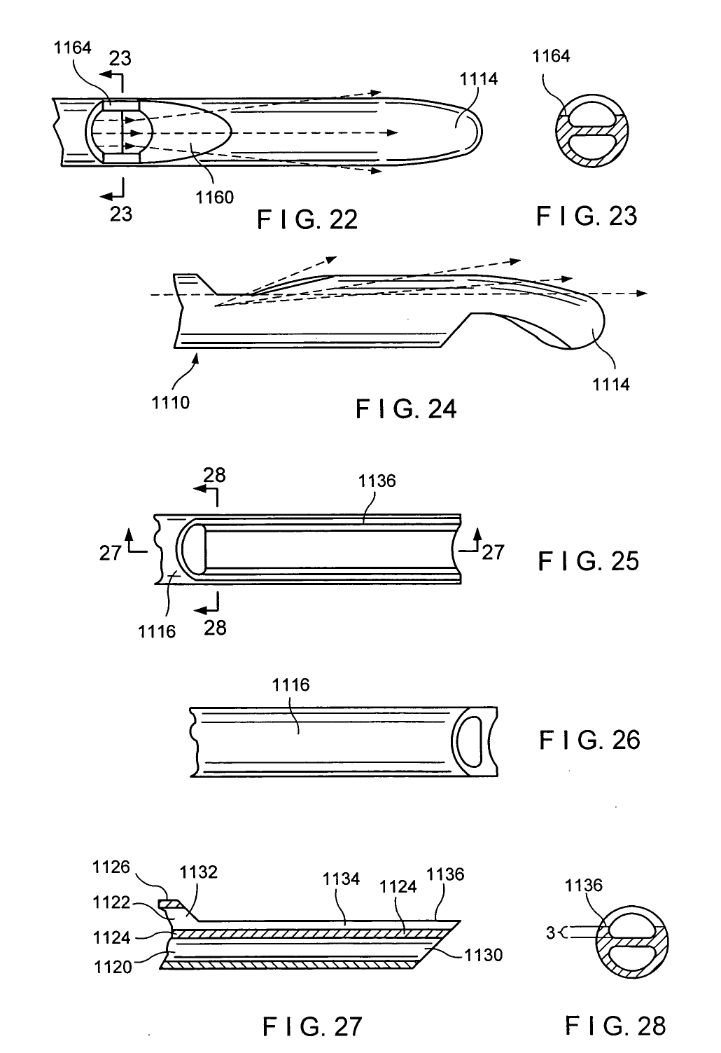

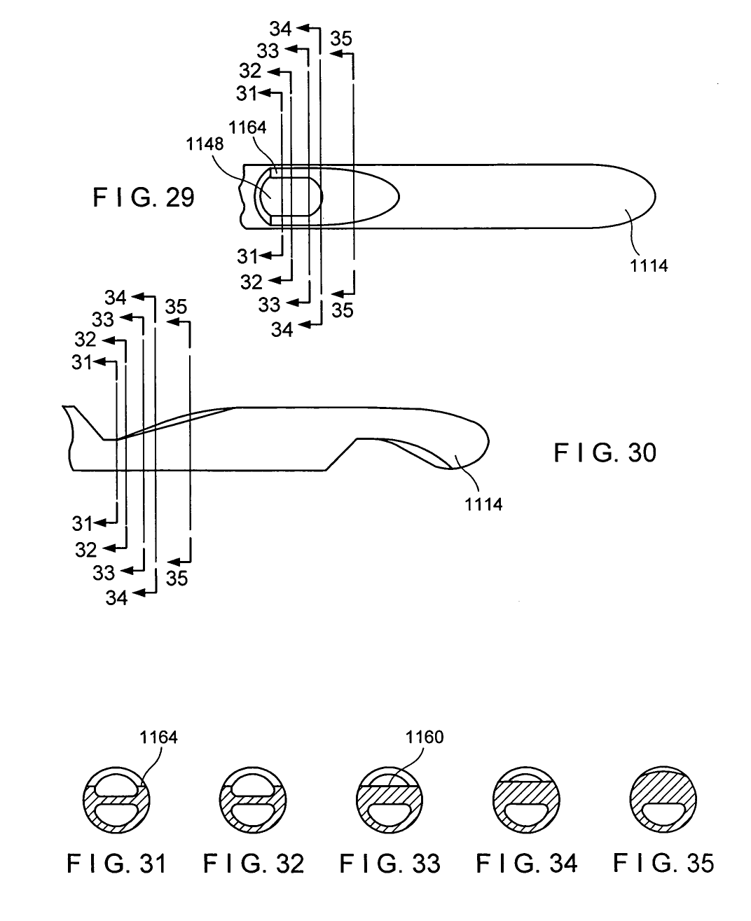

Referring to FIGS. 22-35, the catheter 1110 formed according to the present invention includes a ramp 1160 facing the distal end 1132 of the arterial lumen 1122 and forming an arterial port 1148. The ramp 1160 may be inclined at an angle (e.g., about 21°) relative to the septum 1124. The ramp 1160, where it meets the septum 1124 at a base of the end 1132, may be slightly convex, as best seen in FIG. 32. The ramp 1160 then becomes flat for a substantial (relative) distance, as best seen in FIG. 33. The ramp 1160 then becomes increasingly concave, as best seen in FIGS. 34 and 35, to where it may blend in with a surface of the bolus tip 1114. Adjacent the lumen end 1132 the ramp 1160 is bracketed by an exposed portion 1164 of the side walls 1136.

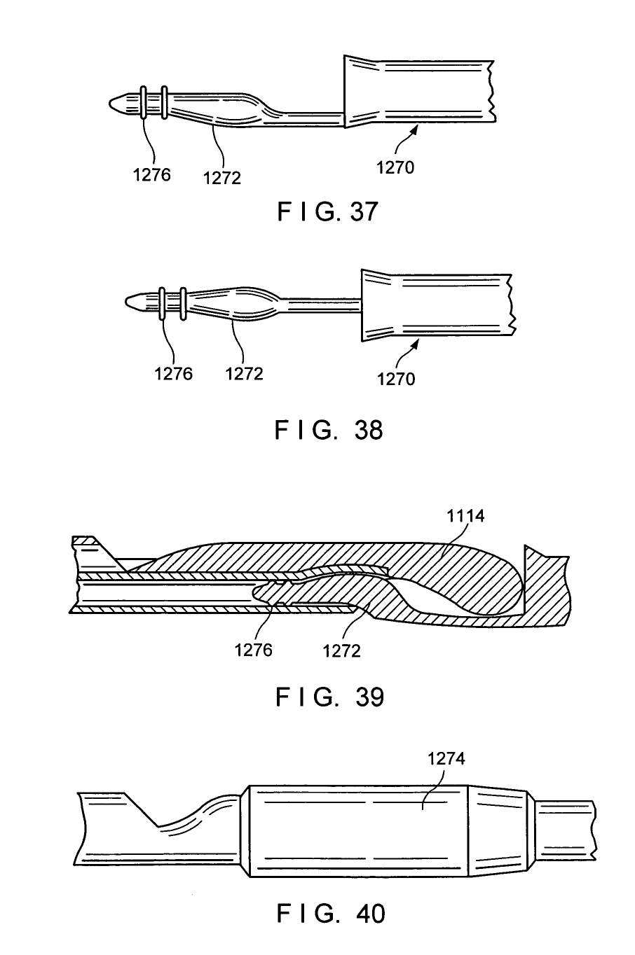

FIGS. 37-40 illustrate a tunneller 1270 and its use in conjunction with a dual lumen catheter and bolus tip according to the present invention. The tunneller 1270 works as a conventional tunneller with a connector probe 1272 being forced into the venous lumen 1122 of the tube 1112. A retention sleeve 1274 may be placed over the tip 1114 and tube 1112 junction to help hold the parts together and to smooth over the transition therebetween. A bulbous section 1272, just behind/proximal of the ribbed portion 1276 that is inserted into the tube 1112, is trapped behind the bolus tip 1114 by the oversleeve 1274 (FIG. 40) to prevent separation of the bolus tip 1114 therefrom.

The side walls 1136 provide certain advantages for the catheter 1110. For example, the side walls 1136 reinforce the catheter 1110 at the arterial port 1148. Downward bending of the bolus tip 1114 is resisted by resistance of the side walls 1136 to stretching. Similarly, upward folding of the bolus tip 1114 is resisted by resistance to axial compression of the side walls 1136.

The ramp 1160, due to its concavity, channels flow (in the reverse flow mode) toward a center of the ramp. Subsequently, the angled section continues to direct flow upward (i.e., radially outward). Finally, the slightly convex ramp section urges flow around the bolus tip 1114 as it proceeds forward over the distal end of the tip. The result is that there is no substantial mixing of flows, i.e., flow directly back toward the venous port.

The present invention provides a dual lumen hemodialysis catheter which accommodates flow rates comparable to separate dual cylindrical lumen tubes and combined dual “D” lumen catheters. The present catheter also allows processed blood to be returned quickly but at a low velocity to avoid tissue damage.

According to the present invention, occlusion of a return line port is substantially avoided regardless of the flow rate and the position of the port in relation to a vessel wall (e.g., a vein wall). However, if port occlusion does occur, it may be relieved by reversing flow through the venous and arterial lumens without greatly increasing the potential for recirculating blood.

In a reverse mode, the arterial port configuration directs flow upward and forward along a ramp angled at approximately 210 relative to an axis of the lumen immediately upon its point of exit from the arterial lumen to direct flow away from the venous port, slow the flow and protect the components of the blood.

A bullet nose may be formed from a predetermined portion of the bolus tip 1114 which is smaller than the outside diameter of the tube to assist in insertion and minimize vessel wall damage. The bullet nose may be inserted using a tunneler and placed in its final location without the utilization of a guide wire. Alternatively, a bolus tip may be formed in place of a prepared distal end of the catheter.

As described above, the catheter tube includes first and second lumens of different lengths. For example, the venous lumen may extend distally beyond the distal end of the arterial lumen leaving the septum between the lumens substantially exposed between those distal ends. The bolus tip which, in itself, may not contain fluid passages, is insert molded onto that exposed septum. The bolus tip may include the bullet nose which extends forward of the distal end of the venous lumen and forms a venous port ramp in front of the venous port. The bolus tip further includes an attachment section which extends forward of the distal end of the arterial lumen and forms an arterial port ramp in front of the arterial port on a side of the catheter opposite the venous port.

The venous port ramp begins at a point where blood exits an ovoid lumen opening (e.g., the venous port) and travels over an ascending arc that slows and directs the flow forward, but also diffuses it, thereby softening the mixing of infused blood with the normal venous flow. In this normal mode, blood is carried forward and away from the aspirating arterial lumen. The ramp is fed by the ovoid lumen opening which is formed in the manufacturing process from the original extruded “D” shape of the tube. This ovoid lumen opening may be slightly larger than the “D”, thereby slowing fluid flow. Its shape, which may be any predetermined shape (e.g., circular, elliptical, square, rectangular, triangular, etc.), may also raise the fluid outflow stream above the normal “D” septum, thereby assisting in the directing the flow up and forward over the top of the bolus tip.

The arterial port ramp may differ from the venous port ramp in several ways. Overall, the arterial port ramp may be longer and, where it begins at the surface of the septum and the opening of the lumen, may be slightly convex in cross-sectional shape. The arterial port ramp may become flat as it continues radially outward and then become slightly convex as it blends into the top surface. In the normal flow mode, the arterial port ramp provides a larger recessed area to allow the maintenance of flow in the reverse mode. In one embodiment, the arterial port ramp has a straight 21° angle ramp profile. However, the ramp angle profile may vary between about 18° and 24°.

In the normal aspiration mode, the rounded top distal end of the arterial port ramp, in cooperation with the top of the inclined edge of the arterial lumen distal end, provides a protected area in the arterial port that assures the continuation of flow in the normal aspiration mode. Those of skill in the art will understand that larger ramp angles may reduce the size of the protected aspiration area, while smaller angles may increase the length and size of the protected aspiration area. However, the additional length increases the tendency of the vessel wall to stretch and protrude into the protected area, thereby reducing its size and presenting the potential for port occlusion. Thus, an angle of approximately 21° is the preferred ramp inclination for aspiration in normal flow, and, in the reverse mode, provides the maximum results for diffusion and flow direction.

Between the bullet nose and the distal end opening of the arterial lumen, short side walls 1136 are formed on the exposed septum. These side walls 1136 serve several purposes controlling fluid flow and stiffening the catheter so that any tendency of the catheter to fold/kink is counteracted. For example, the 45° angle of the proximal edge of the arterial port opens for flow therefrom so that the flow velocity is not increased as blood exits the port. That is, fluid can flow forward and upward without restriction. Similarly, the 21° angle ramp 1160 rises from the floor of the venous port at a point substantially even with a leading edge of the 45° angle arterial lumen opening preventing any increased resistance to flow except by the ramp. Top edges of the side walls 1136 meet the 45° inclined edge of the arterial lumen opening proximal to a junction of the ramp and the surface of the lumen, after the ramp has ascended from the septum surface by an amount equal to a height of the side walls 1136. The side walls 1136 may contain a lower level of the fluid outflow that first meets the resistance of the ramp. As has been explained, the ramp 1160 tends to push flow upward (radially outward), but also tends to diffuse it around the tube. The side walls 1136 reduce the tendency for diffusion at this initial point.

The present invention has been described with reference to specific embodiments, and more specifically to a dialysis catheter with dual lumens. However, other embodiments may be devised that are applicable to different medical devices, without departing from the scope of the invention. Accordingly, various modifications and changes may be made to the embodiments, without departing from the broadest spirit and scope of the present invention as set forth in the claims that follow. The specification and drawings are accordingly to be regarded in an illustrative rather than restrictive sense.

What is claimed is:

1. A multi-lumen catheter comprising:
a first lumen extending through the catheter to a first distal opening;
a second lumen extending through the catheter to a second distal opening which is distal to the first distal opening so that an extending portion of a septum separating the lumens extends distally past the first distal opening; and
a tip overmolded on the extending portion, the tip including a first ramp adjacent to the first distal opening and a second ramp adjacent to the second distal opening, the ramps directing fluids exiting the openings away from a longitudinal axis of the catheter.

2. The catheter according to claim 1, wherein the first distal opening opens at a first angle relative to the septum and the second distal opening opens at a second angle relative to the septum.

3. The catheter according to claim 2, wherein the first angle is approximately 45 degrees and the second angle is approximately 225 degrees.

4. The catheter according to claim 1, wherein the first ramp projects at a first angle relative to the septum and the second ramp projects at a second angle relative to the septum.

5. The catheter according to claim 4, wherein the first angle is between approximately 150 and 175 degrees relative to the septum and the second angle is between approximately 185 and 210 degrees relative to the septum.

6. The catheter according to claim 1, wherein a cross-sectional area of an extension portion of the second lumen distal of the first distal opening is increased relative to a cross-sectional area of a portion of the second lumen proximal to the first distal opening.

7. The catheter according to claim 6, wherein the extension portion is defined by the extending portion of the septum and an outer wall of the catheter, and wherein the extending portion of the septum angles radially outward from a longitudinal axis of the second lumen to increase the cross-sectional area of the extension portion.

8. The catheter according to claim 6, wherein at least portions of the tip are formed integrally with a body of the catheter.

9. A method of forming a distal tip for a multi-lumen catheter comprising:
providing a catheter with a first lumen extending through the catheter to a first distal opening and a second lumen extending through the catheter to a second distal opening distal to the first distal opening so that an extending portion of a septum separating the lumens extends past the first distal opening; and
overmolding on the extending portion a tip including a first ramp adjacent to the first distal opening and a second ramp adjacent to the second distal opening, the first ramp directing fluids from the first distal opening at a first angle relative to a longitudinal axis of the catheter and the second ramp directing fluids exiting the second distal opening at a second angle relative to the longitudinal axis of the catheter.

10. The method according to claim 9, further comprising:
creating the first distal opening by removing at a third angle α first portion of a catheter wall surrounding the first lumen; and
creating the second distal opening by removing at a fourth angle α second portion of the catheter wall surrounding the second lumen.

11. The method according to claim 10, wherein the third angle is approximately 45 degrees relative to the septum and the fourth angle is approximately 225 degrees relative to the septum.

12. The method according to claim 10, further comprising:
expanding the second distal opening so that a cross-sectional area thereof is larger than a cross-sectional area of the second lumen, while maintaining a cross-sectional area of the tip substantially constant.

13. The method according to claim 12, wherein the first ramp projects at the first ramp angle relative to the septum and the second ramp projects at the second ramp angle relative to the septum.

14. The method according to claim 13, wherein the first angle is between approximately 150 and 175 degrees relative to the septum and the second angle is between approximately 185 and 210 degrees relative to the septum.

15. The method according to claim 9, wherein the tip is bonded to the extending portion by one of a mechanical fitting, a friction fitting, chemical bonding and thermal bonding.

16. The method according to claim 9, wherein the first distal opening is separated from the second distal opening by about 1.0 cm to 1.5 cm.

17. A multi-lumen catheter comprising:
a first lumen extending through the catheter to a first distal opening;
a second lumen extending through the catheter to a second distal opening which is distal to the first distal opening so that an extending portion of a septum separating the lumens extends distally past the first distal opening;
a tip formed on the extending portion, the tip including a first ramp adjacent to the first distal opening and a second ramp adjacent to the second distal opening, the ramps directing fluids exiting the openings away from a longitudinal axis of the catheter; and
a pair of side walls disposed on an exposed area of the extending portion between the first distal opening and the first ramp.

18. The catheter according to claim 17, wherein the tip is overmolded on the extending portion.

19. The catheter according to claim 17, wherein the first distal opening is formed at a predetermined angle relative to the extending portion.

20. The catheter according to claim 19, wherein the angle is approximately 45 degrees.

21. The catheter according to claim 20, wherein proximal top edges of the side walls meet the first distal opening proximal to a junction of the tip and the extending portion.

22. The catheter according to claim 21, wherein distal top edges of the side walls meet the tip when the tip has ascended from the extending portion to an amount equal to a height of the side walls.

23. The catheter according to claim 17, wherein the side walls extend from the septum between approximately 0.015 and 0.035 inches.
